(19) United States
(12) Patent Application Publication
(10) Pub. No.: US 2012/0140056 A1
(43) Pub. Date: Jun. 7, 2012

(54) SAMPLE IMAGING SYSTEM AND METHOD FOR TRANSMITTING AN IMAGE OF CELLS OR TISSUES LOCATED IN A CULTURING SPACE TO DATA PRCESSING MEANS

(76) Inventors: Csaba Pribenszky, Budapest (HU); Miklós Molnár, Budapest (HU)

(21) Appl. No.: 13/382,619

(22) PCT Filed: Jul. 9, 2010
(86) PCT No.: PCT/HU10/00081

§ 371 Date: Feb. 20, 2012

(30) Foreign Application Priority Data
Jul. 10, 2009 (HU) P0900431

(51) Int. Cl. H04N 7/18

(52) U.S. Cl. 348/79

(57) ABSTRACT

The invention relates to a sample imaging system (1) for transmitting an image of cells or tissues located in a culturing space (6) to data processing means (7), including a microscope unit (2) intended to be used in the culturing space (6), the microscope unit (2) comprising a frame, an object holder provided on the frame and allowing the cells or tissues to be held substantially immobile during a culturing period, an imaging means arranged on the frame for the optical imaging of the cells or tissues held on the object holder and an image capturing means capturing an image projected by the imaging means. Furthermore, the microscope unit (2) has a connecting means (4) being able to be led out of the culturing space (6) and providing electrical power supply to the microscope unit (2) and transmitting the captured image and the connecting means (4), which connecting means (4) is adapted to be connected to a control unit (3) intended to be used outside of the culturing space (6) and transmitting the captured image to the data processing means (7) and the control unit (3) comprises means adapted to suspend the electrical power supply of the microscope unit (2) with the exception of a period for capturing the image of the cells or tissues and transmitting the captured image to the control unit (3) via the connecting means (4). The invention also relates to a method for transmitting the image of the cells or tissues located in the culturing space (6) to the data processing means (7).

The invention relates to a sample imaging system and a method for transmitting an image of cells or tissues located in a culturing space to data processing means.

Cell or tissue culturing is often necessary for biological, biotechnological or medical procedures or experiments. The term “cells or tissues” in the present description denotes living biological material consisting of one or more cells, including embryos. Although culturing in certain cases can be performed on room temperature, under normal humidity conditions and with a gas composition identical to that of the normal atmospheric air, cells or tissues are often required to be placed in an incubator where the sample is kept on a predetermined temperature and/or in an artificial environment with a predetermined humidity and/or gas composition (e.g. CO₂, O₂ and/or N₂ content) throughout the culturing period. In embryological or artificial insemination laboratories fertilized oocytes and embryos that develop from the cleavage of fertilized oocytes are cultured on a constant temperature of approximately 37° C., with approximately 5% to 6% CO₂ content and approximately 90% relative humidity for a period of 1 to 9 days for example. Other cells or tissues may require different environmental conditions provided by the incubator.

Visual inspection of cells or tissues might not only be necessary at the end of the planned culturing period, but also on several occasions during the culturing. Observation of the dynamics of the embryo development is very important for judging e.g. the viability of the embryos. Removing the cells or tissues from the incubator for repeated observations is so stressful for the cells or tissues that it might hinder the development thereof or even cause their death. Such stress is caused by the removal of cells or tissues from the artificial environment for the time of observation on the one hand and, on the other hand, moving of the cells or tissues itself represent disturbance for them. Therefore several sample imaging devices have been developed for capturing images of cells or tissues repeatedly during the culturing period that do not require the removal of cells or tissues from the incubator in order to enable the user to gain information about the culturing process by the inspection of the resulted images or by processing and analysing the information acquired therefrom.

Document EP 1 548 488 A1 (Tokai Hit Co. Ltd.) discloses a micro-incubator, the closed sample containing chamber of which can be placed on an object holder of a microscope and therefore the user can perform the observation with the microscope at any time or the development of the sample can be recorded with a camera that is connected to the microscope in a conventional way. Since the capsule is connected to the water and CO₂ supplying devices via tubes, the observation of the development of more than one sample can be inconvenient. In addition, the structure of the micro-incubator is extremely complex, and hence the observation of the single inserted sample is very expensive.

Several microscope manufacturer companies produce trinocular inverse microscopes with object holders around which mini-incubators are built. An embryo plate with several wells can be placed on the object holder of the microscope, which enables multiple samples to be observed by moving the plate with a micro-motor. However, the temperature and other parameters as a function of the position in the incubator can vary to a greater degree than in a normal-sized standard laboratory incubator and the number of observable samples is limited, in particular if the built-in microscope of great value is taken into consideration.

Document US 2006/0115892 A1 (YAMAMOTO et al.) discloses an incubator with a rack system arranged therein, capable of storing many sample containing plates. The sample containing plates are taken out by a complex mechanism and moved into the field of view of a sample imaging means that is arranged above one of the sample accommodating portions of the incubator. The incubator transmits the image captured by the sample imaging means to an external computer. Although the observation of many samples is enabled in this case, each sample may be inspected only relatively rarely and as it is noted previously, the sample's movement itself might negatively influence its development. Moreover, the cleaning of the incubator, which is of great importance in order to avoid infection of the samples, is almost impossible with such a sophisticated moving mechanism.

The above mentioned document US 2006/0115892 A1 (YAMAMOTO et al.) also describes an arrangement that has no moving mechanism in the chamber of the incubator but the microscope observation unit is just placed on one of the shelves of the incubator, while the cells or tissues are situated on the microscope observation unit, on the observation window thereof. Within the housing of the microscope observation unit an optical system, a camera and a focusing means are arranged, the focusing means being provided with an electric motor that moves it perpendicularly to the plane of the observation window. Images captured by the camera are transmitted to an external data processing means (i.e. a computer) via a signal cable that runs through the wall of the incubator.

The present Applicant conducted experiments with a microscope being similar to the one described in the above mentioned document US 2006/0115892 Al, comprising within the housing thereof an optical system consisting of an objective, a prism and a projective, a camera and optionally an electrical circuit controlling an illuminating means that illuminates the sample placed on a sample holder window. During the use of the microscope the Applicant noticed that on several occasions the development of the embryos did not correspond to the expectations; the embryos died after few divisions contrary to the fact that movement-related and light-related stress was successfully kept at minimum with the disclosed sample imaging device. The performed examinations (see example 1 below) revealed that the damage to the embryos was caused by the direct and indirect effects of the electrical current carried by the camera and the controlling electronics located inside the device and being under electric tension during the culturing period.

An object of the present invention is to eliminate or at least alleviate the mentioned drawbacks.

This object is achieved by providing a sample imaging system for transmitting an image of cells or tissues located in a culturing space to data processing means as defined in independent claim 1 and by providing a method as defined in independent claim 7. Certain preferred embodiments of the system and that of the method are described in the dependent claims.

The invention will be described in detail below by means of the description of some preferred embodiments thereof, with reference to the appended drawings, in which

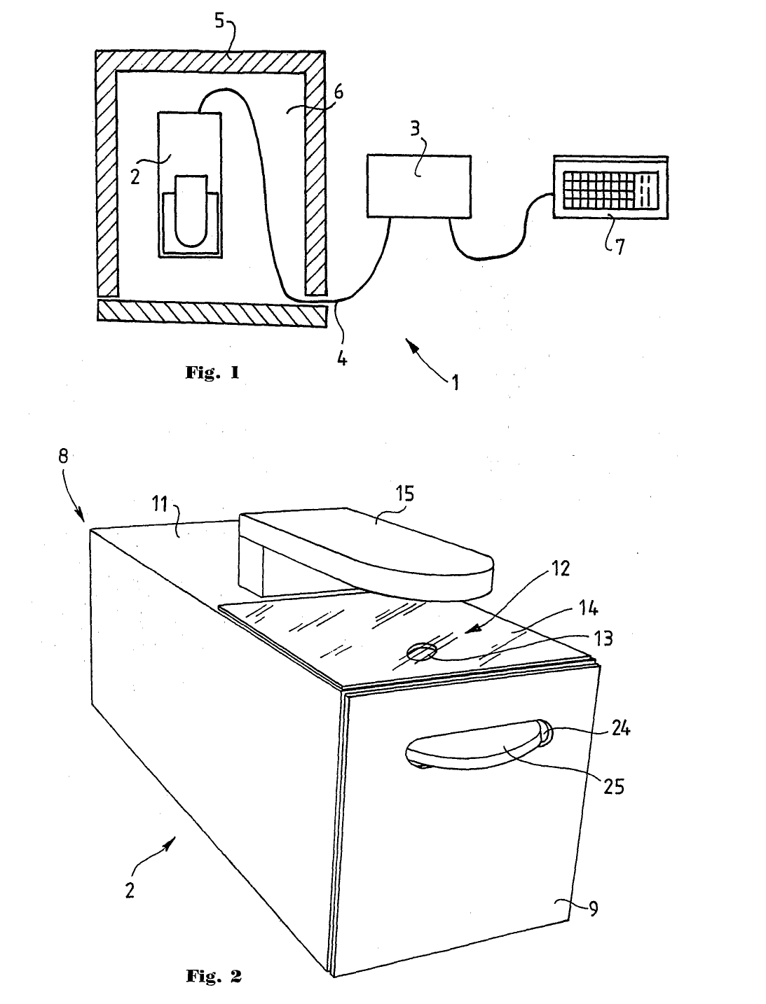

FIG. 1 shows a sectional top view of an embodiment of the sample imaging system according to the invention;

FIG. 2 shows a perspective view of an embodiment of the microscope unit of the sample imaging system according to the invention;

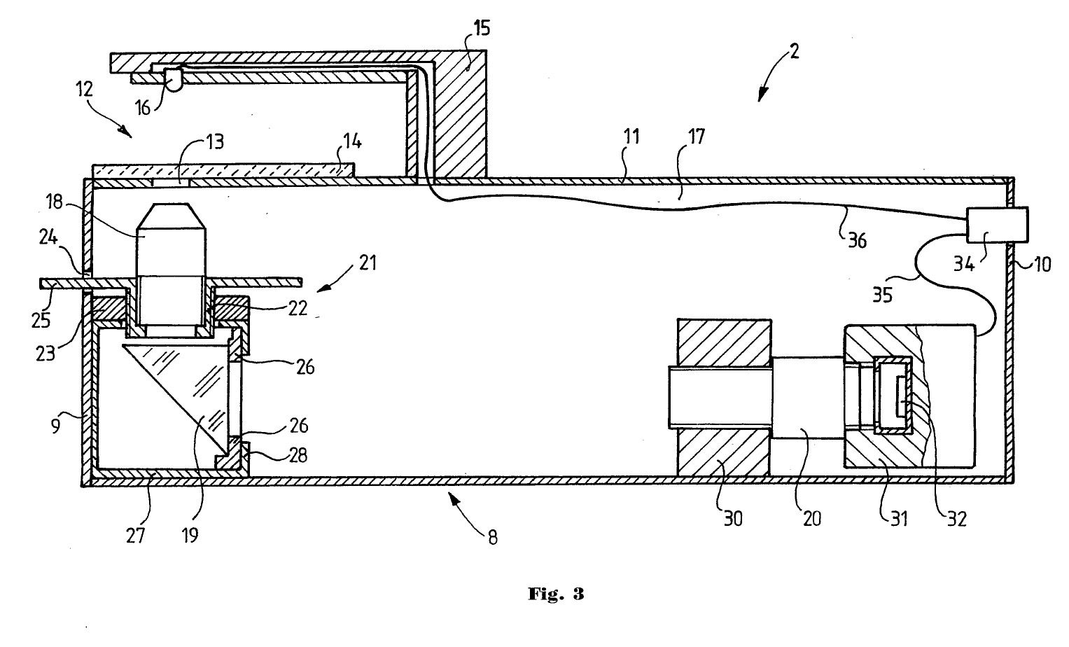

FIG. 3 shows a longitudinal section of the microscope unit shown in FIG. 2; and

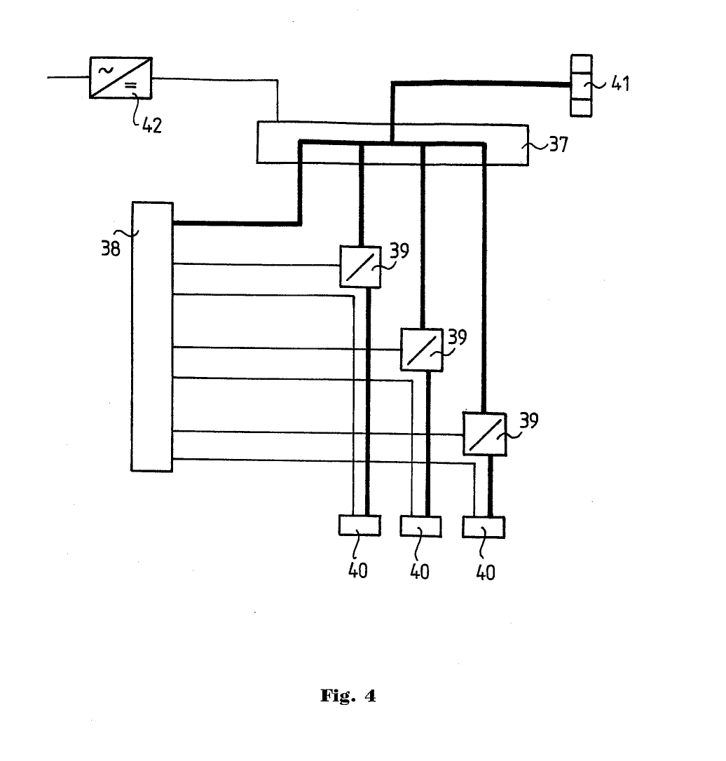

FIG. 4 schematically shows an embodiment of the control unit of the sample imaging system according to the invention.

The same reference signs denote the same elements on the figures.

FIG. 1 shows the sample imaging system 1 of the invention. The sample imaging system 1 consists of two main units: a microscope unit 2 and a control unit 3, connected to each other via a connecting means 4. The microscope unit 2 is intended to be used in a culturing space 6 of an incubator 5 maintaining beneficial environmental conditions for the cultivation of cells or tissues. The microscope unit 2 being placed in the culturing space 6 is used for the imaging of cells, tissues intended to be observed and the transmitting of the captured images via the connecting means 4 to the control unit 3 intended to be used and to be arranged outside the culturing space 6. The control unit 3 in turn transmits the images to a data processing means 7, which is advantageously a notebook computer or other computer means.

FIGS. 2 and 3 show an embodiment of the microscope unit 2 that is capable to be positioned and intended to be used in the culturing space 6 of the incubator 5. The microscope unit 2 has a frame 8 that forms a housing including a hollow profile segment 11 of square cross section, closed by a front plate 9 and a back plate 10. Every element of the frame 8 may be constructed of any corrosion resistant material, preferably from aluminium, stainless steel, plastic—e.g. ABS (acrylonitril-butadiene-styrene)—or even glass, etc. or from an inherently non-corrosion resistant material being made corrosion resistant by surface treatment. Substantially the frame 8, in particular the hollow profile segment 11 is responsible for the mechanical stability of the whole microscope unit 2. The front plate 9 and the back plate 10 may be fixed to the hollow profile segment 11 with e.g. adhesive bonding.

The object holder 12 is provided on the top wall of the hollow profile segment 11, on which the cells or tissues intended to be observed and imaged can be placed and which ensures that the cells or tissues can be held substantially immobile during the culturing period. Therefore an opening or sample window 13 is provided on the top wall of the hollow profile segment 11, which is covered with a plate 14 made of normal glass or, preferably, optical glass or other transparent material, e.g. Plexiglas or polycarbonate. The thickness of the plate 14 can vary between 0.02 mm and 5 mm depending on the working distance of an objective 18, which will be discussed later. Cells or tissues usually kept in sample containers (e.g. Petri dish) can be placed on the top of the plate 14, above the opening 13.

In this embodiment an illumination console 15 is fastened on the top of the frame 8, that extends over the object holder 12, which illumination console 15 is equipped with an illuminating means 16 (e.g. LED) that illuminates the cells or tissues placed onto the object holder 12. The role of the illuminating means 16 is to illuminate the cells or tissues with an illumination power of at least 0.01 lux and its power should preferentially vary between 0.01 W and 5 W. Usually, the wavelength of the light emitted by the illuminating means 16 can be in the wavelength range between 400 nm and 700 nm, although light with a wavelength below or above the visible range (ultraviolet or infrared) could also be necessary in certain cases. In the case of examinations performed with the sample imaging system of the invention, utilizing fluorescent vital cell staining procedures, the spectrum of the luminous source must correspond to the staining material used.

Preferably, those skilled in the art are able to select a subrange of the above-indicated wavelength range that is the most suitable for the cells or tissues in order to minimize the stress caused to the cells or tissues of interest. In order to minimize illumination-related stress, the illuminating means 16 is switched on only for the period of observation or imaging during the culturing process, as it will be discussed in detail later. In the illustrated embodiment the LED directly illuminates the cells or tissues, but other embodiments are also possible where the light of the LED is scattered by a mirror with polished matt surface, and this scattered, diffuse light reaches the cells or tissues. Moreover, additional filters, frosted or diffusing glass can also be introduced into the path of the light.

It is noted that in other embodiments the illumination console 15 with the illuminating means 16 might be omitted from the microscope unit 2 and light required for imaging the cells or tissues can also be provided by a luminous source independent of the sample imaging system 1, e.g. the inner space of the incubator 5 where the microscope unit 2 will be used can also be equipped with an illuminating means. Furthermore, it is also possible to place the illuminating means 16 inside the microscope unit 2 so that the light would illuminate cells or tissues from below. This would result in imaging cells or tissues by means of reflected light instead of transmitted light.

The housing 17 that forms the frame 8 of the microscope unit 2 surrounds a chamber 17 in which an imaging means for the optical imaging of cells or tissues that can be arranged on the object holder 12 and an image capturing means capturing the image projected by the imaging means are arranged.

In this embodiment the imaging means consists of an objective 18 that is positioned below the object holder 12, with its optical axis perpendicular to the plate 14 of the object holder 12, a prism 19 arranged below the objective 18 and a projective 20 that is placed in the path of the light beam from the objective 18 and refracted by the prism 19 by 90 degrees.

The objective 18 is a lens system of a magnification of 1 to 200, preferably of 10 and it is responsible for producing a sharp image of the cells or tissues placed in the field of view at the given magnification. The working distance of the objective 18 shows a relationship with its magnification; the working distance decreases as the magnification increases. In the illustrated embodiment the objective 18 used may be a DIN standard non-fluorinated, strain-free planachromat lens system with a magnification of 10, with a fixed 160 mm tube system and a working distance of approximately 1 cm.

Since the distance between the plate 14 and the cells or tissues placed on the plate 14 may vary depending on the wall thickness of the sample container and also on the location of the cells or tissues within the container, the objective 18 is provided with a focusing means 21 which allows the adjustment of the image sharpness and which is able to move the objective 18 in the direction of its optical axis. The objective 18 is screwed in an objective mount 22 or it can also be fixed there by means of adhesive bonding. The outer surface of the objective mount 22 is provided with a threading with 1 to 4 threads and with a pitch of 0.1 mm to 4 mm, preferably 0.5 mm to 2 mm, most preferably 1 mm. The objective mount 22 is screwed in a focusing holder ring 23 provided with the same threading and it is provided with a focusing wheel 25 that protrudes from the housing through an opening 24 provided on the front plate 9. This enables manual adjustment of the image sharpness by turning the focusing wheel 25 after the cells or tissues are arranged on the plate 14 of the object holder 12. The use of a large focusing wheel 25 enables precise and easy focusing. The height of the opening 24 enables the vertical travel of the focusing wheel 25 which is required for the focusing. A closed design of the housing forming the frame 8 can be attained for example by a focusing wheel that is positioned outside of the housing e.g. on the top wall thereof and which rotates an axle passing through the top wall in a sealed manner and on an inner end of the axle a disc is provided which, in turn, rotates the objective mount 22 by means of a ribbed belt. This design minimizes the penetration of water vapour into the housing from the humid culturfing space 6; in order to absorb the vapour that nevertheless enter the housing and to protect the optical and electronic devices within the microscope unit 2, a silica gel can be arranged inside the housing and replaced in predetermined intervals. In further embodiments, the objective 18 can also be driven by an electric motor or any other way known in the art.

The prism 19 refracts the light from the objective 18 by 90 degrees and hence enables the microscope unit 2 to have a design extending substantially horizontally, which facilitates its positioning in the incubator 5. In the present embodiment the prism 19 is a glass prism sized 22 mm×22 mm×22 mm, with an angle of 45°, which can be replaced by mirrors (e.g. polished metal surfaces) in other embodiments. The prism 19 is cemented to a prism holder 26, which, in turn, is cemented to the wall 28 of the prism housing 27 at an opening on the vertical wall 28 of the prism housing 27; the prism housing 27 is constructed from a hollow profile. An opening is also provided on the top wall 29 of the prism housing 27 into which the objective 18 fixed in the objective mount 22 can protrude, which is held by the focusing holder ring 23 being fixed e.g. by adhesive bonding on the top wall 29. The prism housing 27 itself is fixed to the bottom of the hollow profile segment 11 by screws (not shown) that pass through the hollow profile segment 11 or, alternatively, by means of adhesive bonding.

The light leaving the prism 19 through the opening of the prism holder 26 and the opening of the vertical wall 28 of the prism housing 27 enters the projective 20. In this embodiment the plan-corrected projective 20 that projects a distortion free image onto the image capturing means, is a lens system of a magnification of 0.45. The projective holder 30 of the projective 20 is similarly fixed to the hollow profile segment 11 as is the prism housing 27 i.e. by means of screws (not shown) passing through the bottom of the hollow profile segment 11 or, alternatively, by means of adhesive bonding.

The image capturing means 31 is formed by a sensor 32 that is positioned inside a camera housing 31. The projective 20 and the camera housing 31 are connected to each other by a C-mount thread. The sensor 32 inside the camera housing 31 has another housing 33, which, in this embodiment, is closed by a glass plate in the direction of the incident light. The spectral sensitivity curve of the sensor 32 should overlap the spectrum of the light emitted by the illuminating means 16. The sensor 32 might either be a CCD or a CMOS sensor, with a preferable resolution of at least 1 megapixel, and its size may vary between ¼ inch (6.35 mm) and 1⅛ inch (28.575 mm), it is preferably ½ inch (1.27 mm). It should be noted that the image projected by the projective 20 should advantageously cover the entire surface of the sensor 32. The sensor 32 may be either monochrome or colour, its maximal frame rate is preferably at least 2 images/second and it typically varies between 30 to 60 images/second but at higher frame rates usually it can only be used with lower resolution. The total magnification of the microscope unit 2 at the sensor 32 can be calculated by multiplying the magnifications of the objective 18 with that of the projective 20.

In the present preferred embodiment the sensor 32 can be controlled via a USB port thereof and the captured image can also be transmitted via the same USB port through the said connecting means 4 to the control unit 3. In the illustrated embodiment the USB port of the sensor 32 is connected to a connector 34 mounted onto the back panel 10 of the housing forming the frame 8 by means of a cable 35 and a cable 36 supplying the illuminating means 16 mounted in the illumination console 15 also connects here, which 36 cable partially runs in a channel of the illumination console 15.

In the case of another embodiment where the focusing is performed motorically, the motor would be connected to the connector 34 and image sharpness could be adjusted by means of the control unit 3 or the data processing means 7 even automatically e.g. based on the contrast of the captured image.

Returning to the embodiment of FIG. 2, it will be appreciated that the connector 34 and the connecting means 4 connected thereto not only transmits the captured images towards the control unit 3 and the data processing means 7, but also provides electrical power supply to the microscope unit 2.

FIG. 1 shows that the connecting means 4 connected to the connector 34 of the microscope unit 2 that can be placed into the incubator 5 can be led out of the incubator 5 (e.g. at a door of the incubator 5 or through a sealed opening crossing the wall of the incubator 5 or via interconnected connectors inserted in the wall of the incubator 5 facing both inwards and outwards) and it can be connected to the control unit 3 that can be arranged outside of the incubator 5. On the other hand, the control unit 3 of the sample imaging system 1 can be connected to the data processing means 7.

The control unit 3 performs two tasks. It receives the images captured by the microscope unit 2 and transmits them to the data processing means 7 and it also provides electrical power supply to the microscope unit 2 in such a way that it suspends the electrical power supply of the microscope unit 2 with the exception of a period for capturing the image of the cells or tissues by the image capturing means i.e. the sensor 32 and transmitting the captured image via the connecting means 4 and thus it puts the microscope unit 2 into a voltage free and current free state, which minimizes any harmful effects caused by electrical and/or electronic devices in close proximity to the observed cells and tissues. For this purpose the control unit 3 comprises means suspending the electrical power supply of the microscope unit 2 with the exception of a period for capturing the image of the cells or tissues and transmitting the captured image to the control unit 3 via the connecting means 4.

A schematic diagram of an embodiment of the control unit 3 is shown in FIG. 4. In the illustrated example and in line with what has been said above, the control unit 3 provides electrical power supply to the sensor 32 and the illuminating means 16 in such a way that it suspends the electrical power supply with the exception of the time when the microscope unit 2 is actually used for imaging.

The exemplary control unit 3 includes a four-port USB hub 37, a microscope controlling circuit 38, three solid state switches 39, three connectors 40 for connecting the connecting means 4 of one, two or three microscope units 2, a USB socket 41 for establishing connection with the data processing means 7 and a power supply unit 42 that provides electrical power supply to the control unit 3 and, further, to the microscope units 2 being connected via the connecting means 4 to the connectors 40 by means of the USB hub 37 and the microscope controlling circuit 38.

The USB hub 37 not only establishes connection between the data processing means 7 and the USB devices (in our example the sensors 32) within the one or more microscope units 2 connected to the control unit 3, but it also establishes connection between the data processing means 7 and the microscope controlling circuit 38. The data processing means 7, which is a notebook computer in this example, can thus communicate with the microscope controlling circuit 38 via the USB bus when an image should be taken by the microscope unit 2. Then the microscope controlling circuit 38 sends such a signal to the corresponding one of the three solid state switches 39, which results in connecting the port of the USB hub 37, corresponding to the microscope unit 2 in question to the relevant connector 40. This way the sensor 32 within the microscope unit 2 receives electrical power supply via the USB bus, and this also enables the taking of an image of the cells or tissues placed on the object holder 12 of the given microscope unit 2 via the control unit 3 and the transmining of the image taken to the data processing means 7 through the connecting means 4 and the control unit 3. At the same time the microscope controlling circuit 38 outputs a square wave signal with a variable duty factor and a voltage that exceeds the on voltage of the LED to its output connected to the connector 40 belonging to the given microscope unit 2, to which the illuminating means 16 i.e. the LED of the respective microscope unit 2 is connected via the connecting means 4. It results in the LED illuminating the cells or tissues placed on the object holder 12 with a light intensity corresponding to the duty factor. The data processing means 7 is able to adjust the duty factor and hence the light intensity by means of a command sent to the microscope controlling circuit 38 via the USB bus.

After an image of the cells or tissues have been taken and transmitted to the control unit 3, the microscope controlling circuit 38 disconnects the microscope unit 2 from the USB hub 37 by means of the solid state switch 39 and suspends outputting the square wave signal to the LED. The solid state switch 39 interrupts both the power and the signal leads. This way the microscope unit 2 will not receive either power supply nor signal voltage and therefore it will enter a voltage free and current free state. In this embodiment the microscope controlling circuit 38 and the solid state switch 39 forms such a means, that is adapted to suspend the electrical power supply of the microscope unit 2 with the exception of the period for capturing the image of the cells or tissues and transmitting the captured image to the control unit 3 via the connecting means 4.

The presented embodiment enables the control unit 3 to suspend the power supply of the illuminating means 16 immediately after the image has been captured and when the transmission of the image from the sensor 32 to the control unit 3 is still is progress. This results in further reduction of illumination-related stress to the cells or tissues. As an alternative to this approach, a less complex arrangement would be if the illuminating means 16 i.e. the LED would directly be supplied from the USB port of the sensor 32 via a serial resistance. In this embodiment the control of the light intensity is not possible and nor is the independent switching of the illuminating means 16.

The said solid state switch 39, of course, represents only an example for such a means that enables the disconnection of the microscope unit 2 from the power supply which is the USB bus in the above case.

It will be appreciated that in this example the control unit 3 with the four-port USB hub 37 is able to serve three microscope units 2 and to transfer the images captured by these to a single data processing means 7, however the number of the microscope units 2 can easily be increased by increasing the number of the ports of the USB hub 37.

If the focusing in the microscope unit 2 is performed not manually but electrically, then the operating of the motor connected to the connector 34 of the microscope unit 2 could be performed in such a way that the motor would be without electrical power supply i.e. voltage free and current free with the exception of the period for the imaging and the data transfer, similarly as described above.

It is noted that both the illuminating means 16 and the said electric motor-supported focusing means can be constructed as separate USB devices in the microscope unit 2. In this case a USB hub would be connected to the connector 34 of the microscope unit 2, to which, in turn, the microscope unit's 2 USB devices of different functions would be connected and the control unit 3 would disconnect this USB hub situated in the microscope unit 2 and therethrough all of the USB devices within the microscope unit 2 from the USB hub 37.

It will be appreciated that instead of the described partially USB-based solution those skilled in the art may accomplish the communication between the microscope unit 2 and the data processing means 7 via the control unit 3 in several other ways as long as the electrical power supply of the microscope unit 2 is suspended with the exception of the period for the imaging and the data transfer. The USB bus and the USB socket 41 that form the interface between the control unit 3 and the data processing means 7 can be substituted several other ways, the two units can be connected to each other via e.g. RS232 ports, a Bluetooth connection, a LAN or WLAN network etc.

As an additional option, the control unit 3 and the data processing means 7 can be integrated into one device, which basically would not alter the above described functioning of the control unit 3. One possible example for this would be the integration of the control unit as a PCI card into the computer forming the data processing means 7, or as another example, the previously described USB connection could be established inside a common housing of the integrated control unit 3 and data processing means 7. Such an arrangement could also be treated as if the storing and the processing of the images resulted by the imaging of the cells or tissues would be performed inside the control unit 3 itself.

As another possible solution, the sensor 32 would be an analogue CCD device and the captured image would be transmitted as an analogue video signal to the control unit 3 via the connecting means 4. The digitization of the analogue signal would be performed either here or after the transmission to the data processing means 7 and the analogue CCD device (together with the illumination means 16) would be put in the voltage free state by the control unit 3 with the exception of the period for the imaging and the data transfer.

Furthermore, it is noted that the connecting means 4 between the microscope unit 2 and the control unit 3 can not only be embodied by means of a single cable, but it is also possible that the power supply would be provided by one cable, while the data would travel between the units through a further cable or cables, or even via a wireless connection as long as the electrical power supply of the microscope unit 2 is suspended by the control unit 3 with the exception of the period of the imaging or data transfer.

During the use of the sample imaging system 1 cells or tissues (or even only one cell) placed in a sample container are placed on the object holder 12 of the microscope unit 2, that is above the opening 13 on the plate 14, then the connecting means 4 is connected to the connector 34 and the other end of the connecting means 4 is inserted into one of the available connectors 40 of the control unit 3. Focusing can be performed, if necessary, after the control unit 3 was connected by means of its USB socket 41 to the notebook computer forming the data processing means 7. Then the image as imaged by the sample imaging system 1 is displayed on the notebook computer and the focus is adjusted by means of the focusing wheel 25. The microscope unit 2 together with the cells or tissues is then arranged in the incubator 5 where the cells or tissues rest substantially immobile on the object holder 12 of the microscope unit 2 during the whole culturing period, and the connecting means 4 is led out from the culturing space 6 of the incubator 5. (The order of the preparatory steps described so far can mostly be changed, e.g. it is possible to place the sample container with the cells or tissues on the object holder 12 of the microscope unit 2 that has already been placed in the culturing space 6.)

The microscope unit 2 is switched on by the control unit 3 as a result of a command of the computer forming the data processing means 7 at predetermined points in time or a command of a user at an arbitrarily selected point in time, i.e. in the present embodiment, as has been described above, a square wave signal with variable duty factor is sent to the LED forming the illuminating means 16 which will illuminate the cells or tissues and at the same time a connection is established between the sensor 32 and the USB hub 37 of the control unit 3 by means of the solid state switch 39, which USB hub 37 provides electrical power supply to the sensor 32 on the one hand and sends a command for taking an image on the other hand and subsequently receives the data representing the image resulted by the imaging.

According to the invention, the microscope controlling circuit 38 suspends the power supply of the LED and disconnects both the power supply leads and the signal leads of the sensor 32 from the USB hub 37 by means of the solid state switch 39 after the imaging and the transmitting of the data to the control unit 3, whereby the electrical power supply of the microscope unit 2 is suspended until the beginning of the next imaging cycle.

Preferably the electrical power supply of the microscope unit 2 is suspended in about 10% to 99.999% of the total duration of the cell or tissue culturing period such that, the capturing of the image of cells or tissues and the transmitting of the captured image to the control unit 3 are carried out by the microscope unit 2 in intervals preferably comprised in the range from 1 minute to 1 day, more preferably in the range from 10 minutes to 30 minutes in a duration preferably comprised in the range from 1 second to 1 minute, more preferably in the range from 1 second to 30 seconds. The duration of the imaging and the transmission of the image is highly dependent on the resolution of the image taken. With more frequent imaging information on the development of the cells or tissues with better temporal resolution can be obtained, however the cells or tissues are then exposed to more illumination-, heat-, electrical voltage- and current-related stress. If motion picture is recorded instead of a still image, then the duration of the recording will correspond to the length of the motion picture.

Images will be transmitted from the control unit 3 to the data processing means 7 for storage or arbitrary processing, and images themselves and/or an animation constructed therefrom can be viewed on a screen of the computer and these can also be analyzed by software running on the computer. At the end of the culturing the microscope unit 2 can be removed from the incubator 5 along with the cells or tissues and it can then be cleaned before placing new cells or tissues thereon as needed.

It is noted that the size of the field of view of the microscope unit 2 at the object holder 12 is 0.9 mm×1.1 mm in case of the presented preferred embodiment. Preferably, a sample container may be used for this microscope unit 2, on the bottom of which for example 3×3 or 3×4 wells with a diameter of 100 μm to 300 μm and depth of 150 μm to 300 μm each could be created within the said rectangular area, by pressing with a needle-like pointed tool or by laser ablation. If a sample i.e. cells or tissues (e.g. an embryo) is placed into each of the wells, they will not escape from the field of view of the sample imaging device 1 by floating in the fluid media and an extremely beneficial microenvironment will also be established for their development.

The sample imaging system 1 can successfully and cost efficiently be used as disclosed in large incubators belonging to the standard equipment of e.g. embryological laboratories, enabling the economical and simultaneous observation of many samples.

In addition, it is noted that the optical setup of the microscope unit 2 can be different from the one showed in FIGS. 2 and 3. One of the alternatives has already been mentioned earlier: by omitting the prism 19 a straight beam path can be established, which results in a vertical arrangement of the unit. As another option, a reverse arrangement can also be created by using an objective with a longer working distance; in that case the objective would approach the cells or tissues directly from above and not from below, through the bottom of the sample container.

The invention will now be described by means of an example below.

### EXAMPLE 1

Experiments were conducted to investigate the effects of continuous low voltage (the direct effect of electricity and the heat caused by it) on the early in vitro development of mouse embryos. The purpose of the study was to assess how long-lasting electrical current carried by the microscope unit as a device enabling continuous embryo observation in an artificial embryo culturing space (CO₂ incubator) directly and indirectly influenced the development of embryos placed thereon.

Materials and Methods

Superovulatory Treatment

Day—3, 2 p.m.: 10 IU PMSG was administered to the embryo donor candidate female mice intraperitoneally

Day—1, 2 p.m.: 5 IU hCG was administered to the embryo donor candidate female mice intraperitoneally

Day 0, 8:30 a.m. selection of copulated females by plug inspection

Embryo Washing

Standard surgical isolation of single-cell embryos according to the protocol described in the literature and personal routine, in compliance with the regulations on animal protection. On the first day of the experiment, the fallopian tubes of the donor mice were washed through with a washing liquid (e.g. Flushing Medium, Medicult, Denmark) and the obtained cumulus-oocyte complexes were treated with a 0.5 to 1 mg/ml hyaluronidase (e.g. Sigma-Aldrich, USA) enzyme in order to obtain the purified embryos for the experiment.

In Vitro Cultivation

30 μl drops of EmbryoMax KSOM+AA (Millipore, USA) media were used, covered with LiteOil (LifeGlobal), after a preincubation of at least 6 hours, in an incubator with 6% CO₂ content, 90% relative humidity and 37° C. temperature.

Experimental Setup

During the experiments for certain embryo populations microscope units were used that in addition of the LED forming the illuminating means and a digital camera comprising the sensor comprised further controlling electronics, which in case of the sample imaging system according to the invention is situated in the control unit and therefore outside of the cultivation space, far from the embryos. The location of the controlling electronics, the degree to which the housing that formed the frame of the microscope was closed and the duration for which the microscope was switched on were varied in the case of the different embryo populations as shown in table 1.

TABLE 1

| group | arrangement |
|---|---|
| A | a group placed on a complete, closed microscope unit also |
| | comprising controlling electronics with a camera that was |
| | continuously switched on and with illumination in every |
| | 10 minutes |
| B | a group placed on an open microscope housing that carries |
| | a controlling electronics outside of the hollow profile thereof |
| | with a camera that was continuously switched on and |
| | with illumination in every 10 minutes |
| C | a group directly placed on the controlling electronics |
| D | the controlling electronics was placed outside of the hollow |
| | profile of the microscope unit and outside of the cultivation |
| | space; the group was placed on a closed microscope |
| | housing with a camera and illumination switched on in every |
| | 10 minutes (system and method according to the invention) |
| E | control group of embryos with standard microdrop cultivation |

Results

Table 2 summarizes our results.

TABLE 2

| | n | | | | | |
|---|---|---|---|---|---|---|
| | (number | | embryo development | | | |
| Experimental | of | | 2-cell | | | |
| group | embryos) | repetitions | stadium | % | blastocyst | % |
| A | 144 | 16 | 140 | 97% | 6 | 4% |
| B | 108 | 12 | 103 | 95% | 80 | 74% |
| C | 27 | 3 | 7 | 26% | 1 | 4% |
| D | 234 | 11 | 225 | 96% | 212 | 91% |
| E | 332 | 20 | 308 | 93% | 285 | 86% |

In group “A” a combined negative effect consisting of the direct influence of electric current present in the microscope unit continuously and the effect of the heat emitted by electric and electronic devices (digital camera) was observed: the cells divided in one cycle, but only 4% developed further into the blastocyst stage.

In group “B” the controlling electronics was placed outside of the hollow profile of the microscope unit, but due to the continuous power supply the camera observing the embryos caused a 0.8 to 1.5° C. temperature increase, which was also measurable on the embryo-holding surface of the microscope. The development of the embryos was close to normal, but the percentage of embryos reaching blastocyst stadium was more than 10% below the number observed in the control group.

In the case of group “C” the direct effects of electrical current was observed, which resulted in a less than one-third of embryos being capable of division and only one reaching the blastocyst stadium. The highest embryotoxic effect was observed in this group.

In group “D” the digital camera was also switched off during the periods in between taking the images, and therefore no electrical current was carried by the microscope unit, and the controlling electronics was situated outside of the cultivation space. In this group the best embryo developmental percentage was observed, free of any signs of harmful effects.

Group “E”: control group

As a conclusion we can state that low-voltage electrical current has both a direct and an indirect negative effect on the embryo development and by exploiting the system and method according to the invention these negative effects were successfully eliminated.

The invention was described in detail with regard to its preferred embodiments and those skilled in the art can make several modifications and changes therein without departing from the scope of the invention as defined in the appended claims.

1. A sample imaging system for transmitting an image of cells or tissues located in a culturing space to data processing means, including a microscope unit (2) intended to be used in the culturing space (6), the microscope unit (2) comprising a frame (8), an object holder (12) provided on the frame (8) and allowing the cells or tissues to be held substantially immobile during a culturing period, an imaging means arranged on the frame (8) for the optical imaging of the cells or tissues held on the object holder (12) and an image capturing means capturing an image projected by the imaging means and the microscope unit (2) has a connecting means (4) being able to be led out of the culturing space (6) and providing electrical power supply to the microscope unit (2) and transmitting the captured image, characterized in that the connecting means (4) is adapted to be connected to a control unit (3) intended to be used outside of the culturing space (6) and transmitting the captured image to the data processing means (7) and the control unit (3) comprises means adapted to suspend the electrical power supply of the microscope unit (2) with the exception of a period for capturing the image of the cells or tissues and transmitting the captured image to the control unit (3) via the connecting means (4).

2. A system as claimed in claim 1 characterized in that said means of the control unit (3) suspends the electrical power supply of the microscope unit (2) in about 10% to 99.999% of the total duration of the cell or tissue culturing period.

3. A system as claimed in claim 1 characterized in that the microscope unit (2) is provided with an illuminating means (16) and/or an electrically movable focusing means.

4. A system as claimed in claim 1 characterized in that the control unit (3) is provided with an interface transmitting the captured images to the said data processing means (7).

5. A system as claimed in claim 1 characterized in that the control unit (3) and the said data processing means (7) are integrally provided.

6. A system as claimed in claim 1 characterized in that it includes one or more further microscope units (2) intended to be used in the culturing space (6), the connecting means (4) of which being able to be led out of the culturing space (6), providing electrical power supply to the microscope unit (2) and transmitting the captured image is adapted to be connected to the said control unit (3).

7. A method for transmitting an image of cells or tissues located in a culturing space to data processing means, comprising the steps of
placing the cells or tissues on an object holder (12) of a microscope unit (2) comprising an imaging means for the optical imaging of the cells or tissues placed on the object holder (12) and an image capturing means capturing an image projected by the imaging means,
arranging the microscope unit (2) in the culturing space (6),
leading a connecting means (4) of the microscope unit (2) out of the culturing space (6), the connecting means (4) providing electrical power supply to the microscope unit (2) and transmitting the captured image,
holding the cells or tissues on the object holder of the microscope unit (2) arranged in the culturing space (6) substantially immobile during the culturing period,
characterized in that the method further comprises the steps of
connecting the said connecting means (4) to a control unit (3) arranged outside of the culturing space (6)
capturing an image of the cells or tissues by means of the capturing means and transmitting it to the control unit (3) and, in turn, to the data processing means (7) at selected points in time during the culturing period and
suspending the electrical power supply of the microscope unit (2) with the exception of periods for capturing the image of the cells or tissues and transmitting the captured image to the control unit (3) via the connecting means (4).

8. A method as claimed in claim 7 characterized in that the electrical power supply of the microscope unit (2) is suspended in about 10% to 99.999% of the total duration of the cell or tissue culturing period.

9. A method as claimed in claim 8 characterized in that the capturing of the image of cells or tissues and the transmitting of the captured image to the control unit (3) are carried out by the microscope unit (2) in intervals comprised in the range from 1 minute to 1 day in a duration comprised in the range from 1 second to 1 minute.

10. A method as claimed in claim 7 characterized in that a microscope unit (2) is being used which comprises an illuminating means (16) and/or an electrically movable focusing means.

11. A method as claimed in claim 7 characterized in that the control unit (3) is provided with an interface transmitting the captured images to the data processing means (7), which interface is connected to the data processing means (7).

12. A method as claimed in claim 7 characterized in that a control unit (3) is being used which is integrally provided with the data processing means (7).

13. A method as claimed in claim 7 characterized in that it comprises the steps of placing further cells or tissues on the object holders (12) of one or more further microscope units (2), arranging the one or more microscope units (2) in the culturing space (6), leading the connecting means (4) of the one or more further microscope units (2) out of the culturing space (6) and connecting them to the said control unit (3) arranged outside the culturing space (6), the connecting means (4) providing electrical power supply to the one or more further microscope units (2) and transmitting the captured image.
